(19) United States
(12) Patent Application Publication
(10) Pub. No.: US 2022/0054750 A1
(43) Pub. Date: Feb. 24, 2022

(54) BIOLOGICS DELIVERY SYSTEM AND MODULAR TIP DESIGN

(71) Applicant: Ethicon, Inc., Somerville, NJ (US)

(72) Inventor: Jianxin GUO, Livingston, NJ (US)

(21) Appl. No.: 16/998,769

(22) Filed: Aug. 20, 2020

(51) Int. Cl. A61M 5/19; A61M 5/315; A61M 5/32

(52) U.S. Cl. CPC A61M 5/19; A61M 5/3286; A61M 5/31505; A61M 5/31596

(57) ABSTRACT

A method and apparatus for sealing a biologics delivery system, said applicator including two adjoining syringes, each having a barrel and an exit tube distal to the barrel, and an interchangeable delivery tip, having a delivery tip connector portion, and storage cap, each connectable to the exit tube of each barrel, and having a fixed internal O-ring for fluid-tight connection between the delivery tip and storage cap and said exit tube.

### FIELD

This invention relates to a biologics delivery system having modular delivery tips, which resists leakage of the biological fluids.

### ENVIRONMENT

In recent years, minimally invasive surgical techniques have emerged as an alternative to conventional surgical techniques to perform a plurality of surgical procedures. Minimally invasive procedures differ from conventional surgical procedures in that a plurality of devices may be introduced into the body through a small incision. As a result, trauma to the body is greatly reduced, thereby decreasing the recovery time of the patient.

One example of a common minimally invasive surgery involves laparoscopic surgical procedures. Laparoscopic procedures may be used to treat hernias, colon dysfunctions, gastroesophageal reflux disease, gallbladder disorders, etc. Typically, the patient undergoing the procedures will return home hours after undergoing surgery.

One challenge presented when performing minimally invasive surgical procedures relates to reducing bleeding at a surgical site when control of bleeding by standard surgical techniques, such as suturing, ligature and cautery, is ineffective or impractical. As opposed to conventional surgical procedures, the surgeon's access to the site of the incision is greatly reduced during minimally invasive procedures and conventional techniques for hemostasis may be difficult to effect.

Recently, the use of tissue sealants and other biological adhesive materials has emerged as an alternate technique for hemostasis. Such tissue sealants may include fibrin, which is comprised of co-reactive thrombin and fibrinogen materials, although other multiple component materials are available. Typically, the individual components of the sealant materials are stored in isolated reservoirs. When mixed, these components may coagulate very quickly, yielding a gel within a short period of time, perhaps 10 or 20 seconds. When applied to the exterior of the body, or when considerable access to the application site is possible, the rapid coagulative properties of the tissue sealant are advantageous. However, such fast-acting properties of conventional tissue sealants have presented potential problems of fouling or clogging during application through laparoscopic devices, which typically results in the destruction of the device.

To protect against cross-contamination, multi-barrel biologics delivery systems have been developed wherein each reactive material is loaded and sealed in a separate vessel, and then brought together into a unified device, such as a mixing/deliver tip, at the time of use without having to transfer the fluid materials into a separate vial or barrel.

Typically, the syringes of such systems are provided with Luer-type connectors at the distal ends of the syringes. While luer-type syringe connectors are conventional throughout the health care industry, they do not always provide a leak tight seal against fluid :leakage. Additionally, they require a twisting motion to connect and seal with the threads of a conforming device to be attached to the syringe. When side-by-side biologics delivery systems are employed, the twisting motion for locking the leer threads can be awkward.

It would be most convenient to have a syringe/delivery tip design which not only provides improved resistance to liquid leakage, as compared to luer-type connections, but also negates the necessity of the twisting motion necessary to connect the syringes to the delivery tips.

### SUMMARY

Provided is a biologics delivery system, comprising two adjoining syringes, each having a barrel and an exit tube distal to the barrel, and an interchangeable delivery tip, comprising a delivery tip connector portion, and storage cap connectable to the exit tube of each barrel, each having a fixed internal O-ring seal for fluid-tight connection between said delivery tip and storage cap and said exit tube.

In one form, the exit tube does not have a luer connection.

In another form, the biologics delivery system further comprises a locking tab releasably connecting the syringe to the storage cap and delivery tip, such as wherein the locking tab is a flexible tab disposed on the syringes and having a pawl on a distal end thereof to connect to conforming slots in the storage cap and delivery tip connector.

Advantageously, the delivery tip is configured to match the syringes in only one configuration, such as wherein a blocking element located inside the delivery tip connector or storage cap prevents improper connection with the syringe barrels.

In yet another form, the biologics delivery system further comprises a plunger bridge between plungers of said syringes.

In yet another form, the biologics delivery system further comprises a frame to contain said syringes. Advantageously, the frame can comprise a flexible locking tab having a pawl on a distal end thereof and the syringes can be disposed in said frame. In some forms, the frame and syringes can comprise a single part.

In another form, the delivery tip connector can further comprise a manifold.

Additionally presented herein is a method of sealing a biologics delivery system, said system comprising two adjoining syringes, each having a barrel and an exit tube distal to the barrel, said method comprising interchanging a storage cap with a delivery tip having a delivery tip connector portion, each of said storage cap and delivery tip being connectable to the exit tube of each barrel and each having a fixed internal O-ring for fluid-tight connection between said storage cap, said delivery tip and said exit tube.

In one form, the exit tube does not have a luer connection.

In another form, the method further comprises a locking tab releasably connecting the syringe to the storage cap and delivery tip, such as wherein the locking tab is a flexible tab disposed on the syringes and having a pawl on a distal end thereof to connect to conforming slots in the storage cap and delivery tip connector.

Advantageously, the delivery tip is configured to match the syringes in only one configuration, such as wherein a blocking element located inside the delivery tip connector or storage cap prevents improper connection with the syringe barrels.

In yet another form, the method further comprises a plunger bridge between plungers of said syringes.

In yet another form, the method further comprises a frame to contain said syringes. Advantageously, the frame can comprise a flexible locking tab having a pawl on a distal end thereof and the syringes can be disposed in said frame.

### BRIEF DESCRIPTION OF THE DRAWINGS

The present disclosure is susceptible to various modifications and alternative forms, specific exemplary implementations thereof have been shown in the drawings and are herein described in detail. It should be understood, however, that the description herein of specific exemplary implementations is not intended to limit the disclosure to the particular forms disclosed herein.

This disclosure is to cover all modifications and equivalents as defined by the appended claims. It should also be understood that the drawings are not necessarily to scale, emphasis instead being placed upon clearly illustrating principles of exemplary embodiments of the present invention. Moreover, certain dimensions may be exaggerated to help visually convey such principles. Further where considered appropriate, reference numerals may be repeated among the drawings to indicate corresponding or analogous elements. Moreover, two or more blocks or elements depicted as distinct or separate in the drawings may be combined into a single functional block or element. Similarly, a single block or element illustrated in the drawings may be implemented as multiple steps or by multiple elements in cooperation.

The forms disclosed herein are illustrated by way of example, and not by way of limitation, in the figures of the accompanying drawings and in which like reference numerals refer to similar elements and in which:

FIG. 1A presents a perspective view of the biologics delivery system of the present disclosure having a storage cap installed on the distal end thereof;

FIG. 1B presents a perspective view of the biologics delivery system of the present disclosure having a delivery tip installed on the distal end thereof;

FIG. 2A presents a cutaway view of the storage cap according to FIG. 1A;

FIG. 2B presents a cutaway view of the delivery tip according to FIG. 1B;

FIG. 3 presents the biologics delivery system of FIGS. 1A and 1B having both the interchangeable storage cap and delivery tip removed;

FIG. 4 presents a perspective view of the delivery tip connector portion of the presently disclosed delivery tips;

FIG. 5 presents a cutaway view of the delivery tip connector of FIG. 4; and

FIG. 6 presents overall views of several suitable, interchangeable (or modular) delivery tips for use with the biologics delivery system of the present disclosure

### DETAILED DESCRIPTION

Various aspects will now be described with reference to specific forms selected for purposes of illustration. It will be appreciated that the spirit and scope of the apparatus, system and methods disclosed herein are not limited to the selected forms. Moreover, it is to be noted that the figures provided herein are not drawn to any particular proportion or scale, and that many variations can be made to the illustrated forms.

Each of the following terms written in singular grammatical form: “a,” “an,” and “the,” as used herein, may also refer to, and encompass, a plurality of the stated entity or object, unless otherwise specifically defined or stated herein, or, unless the context clearly dictates otherwise. For example, the phrases “a device,” “an assembly,” “a mechanism,” “a component,” and “an element,” as used herein, may also refer to, and encompass, a plurality of devices, a plurality of assemblies, a plurality of mechanisms, a plurality of components, and a plurality of elements, respectively.

Each of the following terms: “includes,” “including,” “has,” “‘having,” “comprises,” and “comprising,” and, their linguistic or grammatical variants, derivatives, and/or conjugates, as used herein, means “including, but not limited to.”

Throughout the illustrative description, the examples, and the appended claims, a numerical value of a parameter, feature, object, or dimension, may be stated or described in terms of a numerical range format. It is to be fully understood that the stated numerical range format is provided for illustrating implementation of the forms disclosed herein and is not to be understood or construed as inflexibly limiting the scope of the forms disclosed herein.

Moreover, for stating or describing a numerical range, the phrase “in a range of between about a first numerical value and about a second numerical value,” is considered equivalent to, and means the same as, the phrase “in a range of from about a first numerical value to about a second numerical value,” and, thus, the two equivalently meaning phrases may be used interchangeably.

It is to be understood that the various forms disclosed herein are not limited in their application to the details of the order or sequence, and number, of steps or procedures, and sub-steps or sub-procedures, of operation or implementation of forms of the method or to the details of type, composition, construction, arrangement, order and number of the system, system sub-units, devices, assemblies, sub-assemblies, mechanisms, structures, components, elements, and configurations, and, peripheral equipment, utilities, accessories, and materials of forms of the system, set forth in the following illustrative description, accompanying drawings, and examples, unless otherwise specifically stated herein. The apparatus, systems and methods disclosed herein can be practiced or implemented according to various other alternative forms and in various other alternative ways.

It is also to be understood that all technical and scientific words, terms, and/or phrases, used herein throughout the present disclosure have either the identical or similar meaning as commonly understood by one of ordinary skill in the art, unless otherwise specifically defined or stated herein. Phraseology, terminology, and, notation, employed herein throughout the present disclosure are for the purpose of description and should not be regarded as limiting.

In the figures, like numerals denote like, or similar, structures and/or features; and each of the illustrated structures and/or features may not be discussed in detail herein with reference to the figures. Similarly, each structure and/or feature may not be explicitly labeled in the figures; and any structure and/or feature that is discussed herein with reference to the figures may be utilized with any other structure and/or feature without departing from the scope of the present disclosure.

Provided herein are new two-component biologics delivery system and delivery tip designs to eliminate the luer connections, which require significant force to seal and are also prone to leak. The modular design for the delivery tips enables all the tip designs with different applications to use the same connection and components to simplify the manufacturing process. The designs also reduce the potential leak interfaces to reduce the likelihood of leaking during application and have features to prevent cross-contamination when users try to detach and reattach tips. The designs also reduce the attachment force between syringes and delivery tips to improve usability.

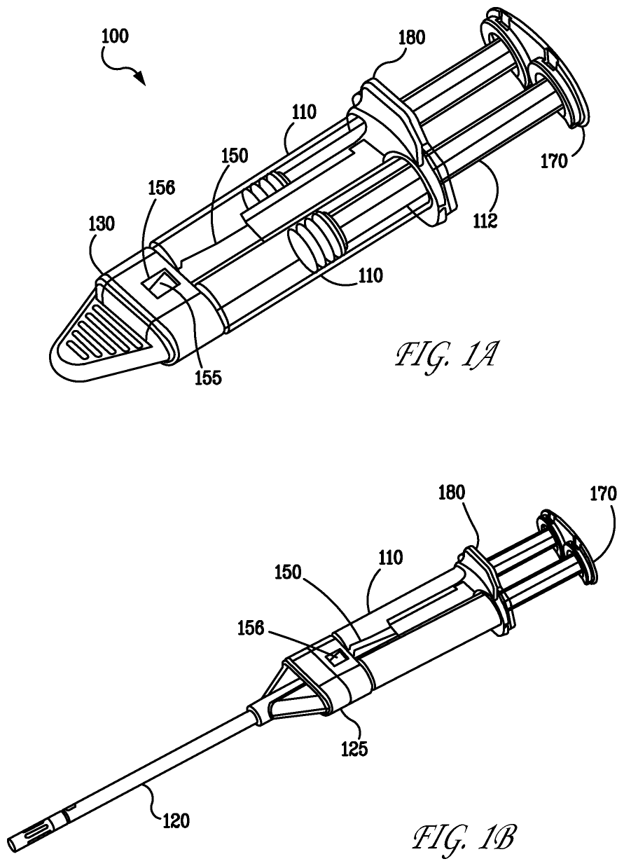

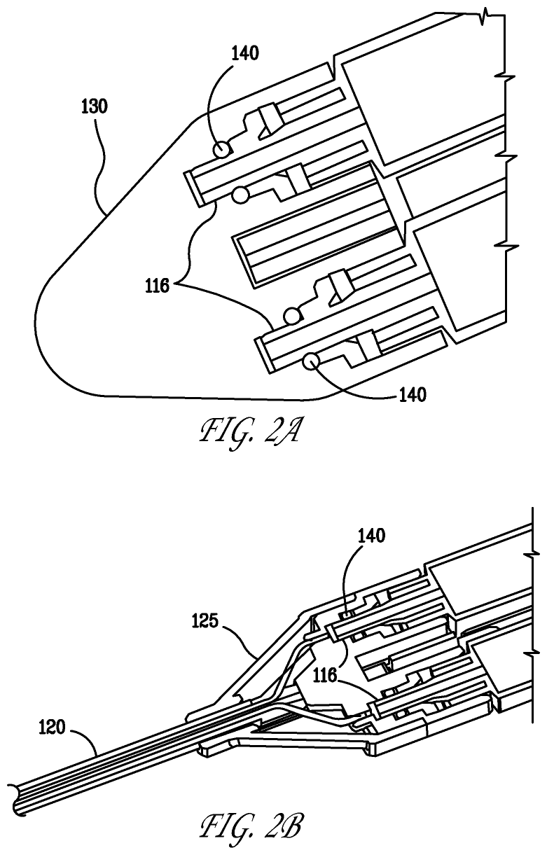

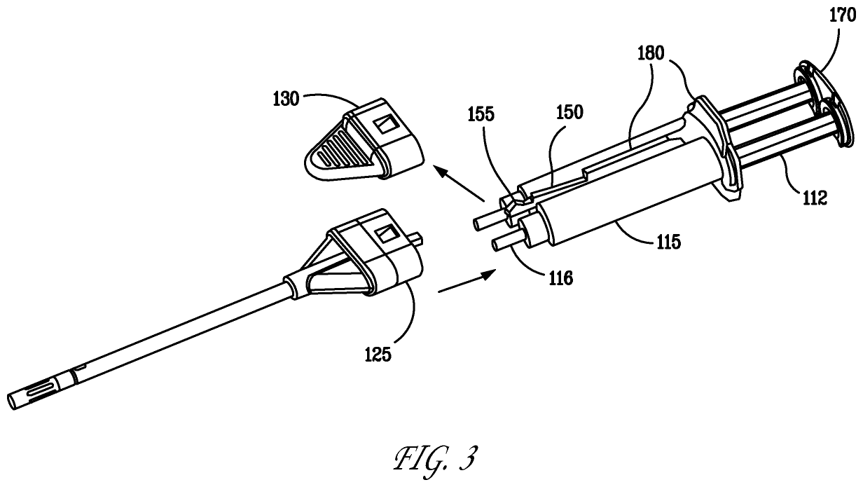

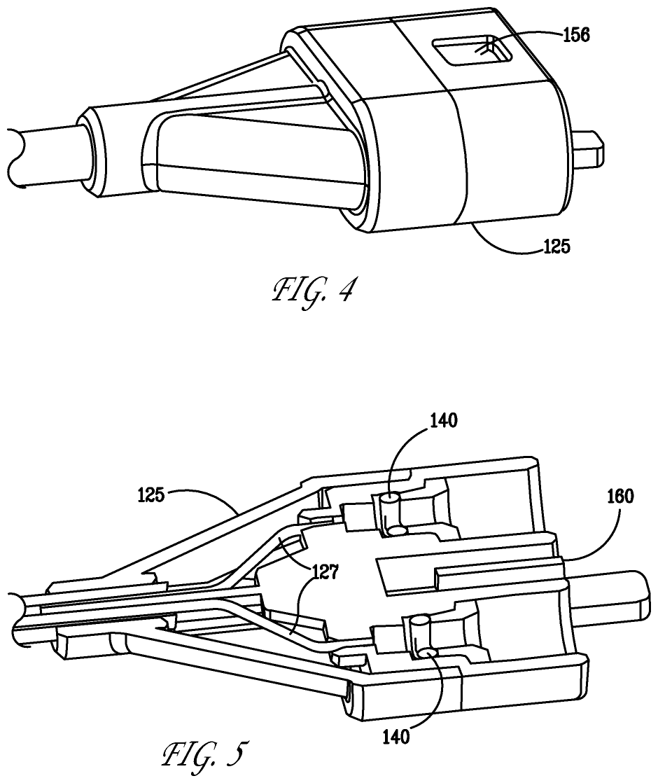

FIGS. 1A through 6 illustrate a biologics delivery system 100, comprising two adjoining syringes 110, each having a barrel 115 and an exit tube 116 distal to the barrel 115, and an interchangeable delivery tip 120, comprising a delivery tip connector portion 125, and storage cap 130 connectable to the exit tube 116 of each barrel, each having a fixed internal O-ring seal 140 for fluid-tight connection between said delivery tip 120, storage cap 130 and said exit tube 116. Conventionally, the syringes 110 incorporate plungers 112 to force the multiple sealing components through the syringes, into the delivery tip 120 and to the surgical situs.

Advantageously, the exit tube 116 portion does not have a luer connection but is instead sealed by application of force against the O-rings 140 disposed within the delivery tip connector 125, and alternatively those disposed within the storage cap 130. The force necessary to seal the O-rings against the exit tube portions of the syringes is considerably less than that necessary to achieve sealing with a luer-type connector.

Once the delivery tip connector 125 is forced against the exit tubes 116, the biologics delivery system 100 has a locking tab 150 releasably connecting the syringe to the storage cap 130 and delivery tip 120, such as wherein the locking tab 150 is a flexible tab disposed on the syringes 110 and having a pawl 155 on a distal end thereof to connect to conforming slots 156 in the storage cap 130 and delivery tip connector 125.

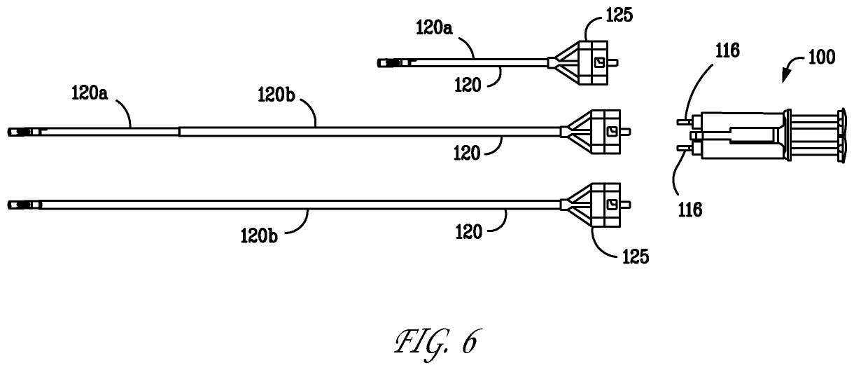

The delivery tip 120 and storage cap 130 are configured to match the biologics delivery system 100 in only one configuration, such as wherein a blocking element 150 located inside the delivery tip connector 125 or storage cap 130 prevents improper connection with the syringe barrels. The blocking element 160 can be configured as an axially off-set, matching key/keyway between the biologics delivery system 100 and the delivery tip connector 125 and storage cap 130, as can be understood from FIG. 5. Other such blocking configurations are known in the art and could be suitably incorporated into the disclosed system.

Advantageously, the biologics delivery system can include a plunger bridge between plungers 112 of said syringes 110, to facilitate consistent delivery from each syringe 110 during use. Additionally, the biologics delivery system can include a frame 180 to contain said syringes 110 in adjoining relationship. Frame 180 can be configured to include the flexible locking tab 150 and pawl 155 on a distal end thereof.

The delivery tip connector can include a manifold 127 therein, to direct the flow of the individual sealing components, such as thrombin and fibrinogen, together and into the delivery tip 120. The presently disclosed connection system is modular, permitting interchangeability between a variety of delivery tips such as open malleable tips 120a, rigid lap tips 120b, and combinations thereof, wherein the tip has both a malleable 120a and rigid 120b portion.

Additionally presented herein is a method of sealing a biologics delivery system 100, said system comprising two adjoining syringes 110, each having a barrel 115 and an exit tube 116 distal to the barrel, said method comprising interchanging a storage cap 130 with a delivery tip 120 having a delivery tip connector portion 125, each of said storage cap 130 and delivery tip 120 being connectable to the exit tube 116 of each barrel 115 and each having a fixed internal O-ring 140 for fluid-tight connection between said storage cap 130, said delivery tip 120 and said exit tube 116. According to the disclosed method, the exit tubes do not have luer connections, which are replaced by the O-ring sealing system.

The method provides for a locking tab 150 releasably connecting the biologics delivery system 100 to the storage cap 130 and delivery tips 120. The locking tab 150 can be a flexible tab disposed on the syringes 110 and having a pawl 155 on a distal end thereof to connect to conforming slots 156 in the storage cap 130 and delivery tip connector 125. In one form, the method provides a frame 180 to contain said syringes 110, into which is provided the flexible locking tab 150 and pawl 155.

The delivery tip 120 is provided with a way to prevent improper connection with the syringe barrels 115, so as to match the biologics delivery system 100 in only one configuration, such as wherein a blocking element 160 located inside the delivery tip connector 125 or storage cap 130 prevents improper connection with the syringe barrels.

Advantageously, the biologics delivery system can include a plunger bridge between plungers 112 of said syringes 110, to facilitate consistent delivery from each syringe 110 during use.

### EXAMPLES

PCT1. A biologics delivery system, comprising: two adjoining syringes, each having a barrel and an exit tube distal to the barrel; and an interchangeable delivery tip, comprising a delivery tip connector portion, and cap connectable to the exit tube of each barrel, each having a fixed internal O-ring seal for fluid-tight connection between said delivery tip and cap and said exit tube.

PCT2. The biologics delivery system of paragraph PCT1, wherein the exit tube does not have a luer connection.

PCT3. The biologics delivery system of paragraphs PCT1 or PCT2, further comprising a locking tab releasably connecting the syringes to the cap and delivery tip.

PCT4. The biologics delivery system of paragraph PCT3, wherein the locking tab is a flexible tab disposed on the syringes and having a pawl on a distal end thereof to connect to conforming slots in the cap and delivery tip connector.

PCT5. The biologics delivery system of any preceding paragraph 1-4, wherein the delivery tip is configured to match the syringes in only one configuration.

PCT6. The biologics delivery system of paragraph 5, wherein a blocking element located inside the delivery tip connector or cap prevents improper connection with the syringe barrels.

PCT7. The biologics delivery system of any preceding claim, further comprising a plunger bridge between plungers of said syringes.

PCT8. The biologics delivery system of any preceding claim, further comprising a frame to contain said syringes.

PCT9. The biologics delivery system of any preceding paragraph 1-8, wherein the delivery tip connector portion further comprises a manifold.

PCT10. The biologics delivery system of paragraph 8, wherein the frame comprises a flexible locking tab having a pawl on a distal end thereof and the syringes are disposed in said frame.

PCT11. A method of sealing a biologics delivery system, said system comprising two adjoining syringes, each having a barrel and an exit tube distal to the barrel, said method comprising interchanging a storage cap with a delivery tip having a delivery tip connector portion, each of said storage cap and delivery tip being connectable to the exit tube of each barrel and each having a fixed internal O-ring for fluid-tight connection between said storage cap, said delivery tip and said exit tube.

PCT12. The method of paragraph 11, wherein the exit tube does not have a luer connection.

PCT13. The method of paragraphs PCT11 or PCT12, further comprising a locking tab releasably connecting the syringes to the cap and delivery tip.

PCT14. The method of paragraph 13, wherein the locking tab is a flexible tab disposed on the syringes and having a pawl on a distal end thereof to connect to conforming slots in the cap and delivery tip connector.

PCT15. The method of any one of paragraphs PCT11-PCT14, wherein the delivery tip is configured to match the syringes in only one configuration.

PCT16. The method of paragraph PCT15, wherein a blocking element located inside the delivery tip connector or cap prevents improper connection with the syringe barrels.

PCT17. The method of any one of paragraphs PCT11-PCT16, further comprising a plunger bridge between plungers of said syringes.

PCT18. The method of any one of paragraphs PCT11-PCT17, further comprising a frame to contain said syringes.

PCT19. The method of any one of paragraphs PCT11-PCT18, wherein the delivery tip connector portion further comprises a manifold.

PCT20. The method of paragraph PCT18, wherein the frame comprises a flexible locking tab having a pawl on a distal end thereof and the syringes are disposed in said frame.

### INDUSTRIAL APPLICABILITY

The systems and methods disclosed herein are applicable to the medical device industry.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and sub-combinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and sub-combinations that are directed to one of the disclosed inventions and are novel and non-obvious. Inventions embodied in other combinations and sub-combinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

What is claimed:

1. A biologics delivery system, comprising:
two adjoining syringes, each having a barrel and an exit tube distal to the barrel; and
an interchangeable delivery tip, comprising a delivery tip connector portion, and storage cap connectable to the exit tube of each barrel, each having a fixed internal O-ring seal for fluid-tight connection between said delivery tip and storage cap and said exit tube.

2. The biologics delivery system of claim 1, wherein the exit tube does not have a luer connection.

3. The biologics delivery system of claim 1, further comprising a locking tab releasably connecting the syringes to the storage cap and delivery tip.

4. The biologics delivery system of claim 3, wherein the locking tab is a flexible tab disposed on the syringes and having a pawl on a distal end thereof to connect to conforming slots in the storage cap and delivery tip connector.

5. The biologics delivery system of claim 1, wherein the delivery tip is configured to match the syringes in only one configuration.

6. The biologics delivery system of claim 5, wherein a blocking element located inside the delivery tip connector or storage cap prevents improper connection with the syringe barrels.

7. The biologics delivery system of claim 1, further comprising a plunger bridge between plungers of said syringes.

8. The biologics delivery system of claim 1, further comprising a frame to contain said syringes.

9. The biologics delivery system of claim 1, wherein the delivery tip connector portion further comprises a manifold.

10. The biologics delivery system of claim 8, wherein the frame comprises a flexible locking tab having a pawl on a distal end thereof and the syringes are disposed in said frame.

11. A method of sealing a biologics delivery system, said system comprising two adjoining syringes, each having a barrel and an exit tube distal to the barrel, said method comprising interchanging a storage cap with a delivery tip having a delivery tip connector portion, each of said storage cap and delivery tip being connectable to the exit tube of each barrel and each having a fixed internal O-ring for fluid-tight connection between said storage cap, said delivery tip and said exit tube.

12. The method of claim 11, wherein the exit tube does not have a luer connection.

13. The method of claim 11, further comprising a locking tab releasably connecting the syringes to the storage cap and delivery tip.

14. The method of claim 13, wherein the locking tab is a flexible tab disposed on the syringes and having a pawl on a distal end thereof to connect to conforming slots in the storage cap and delivery tip connector.

15. The method of claim 11, wherein the delivery tip is configured to match the syringes in only one configuration.

16. The method of claim 15, wherein a blocking element located inside the delivery tip connector or storage cap prevents improper connection with the syringe barrels.

17. The method of claim 11, further comprising a plunger bridge between plungers of said syringes.

18. The method of claim 11, further comprising a frame to contain said syringes.

19. The method of claim 11, wherein the delivery tip connector portion further comprises a manifold.

20. The method of claim 18, wherein the frame comprises a flexible locking tab having a pawl on a distal end thereof and the syringes are disposed in said frame.
